(19) United States
(12) Patent Application Publication
(10) Pub. No.: US 2008/0281365 A1
(43) Pub. Date: Nov. 13, 2008

(54) NEURAL SIGNAL DUTY CYCLE

(76) Inventors: Katherine S. Tweden, Mahtomedi, MN (US); Richard R. Wilson, Arden Hills, MN (US); Christopher C. Pulling, Princeton, MN (US)

Correspondence Address: MERCHANT & GOULD PC, P.O. BOX 2903, MINNEAPOLIS, MN, 55402-0903, US

(21) Appl. No.: 11/746,286

(22) Filed: May 9, 2007

(51) Int. Cl. A61N 1/05

(52) U.S. Cl. 607/2

(57) ABSTRACT

A disorder (e.g., obesity) is treated by applying an electrical signal to an autonomic nerve (e.g., a vagus or splanchnic nerve). The treatment includes applying a signal to a nerve of a patient to be treated. The signal has a duty cycle including an ON time during which the signal is applied by to the nerve followed by an OFF time during the signal is not applied to the nerve. The ON time is selected to have a duration preferably greater than 30 seconds and up to 180 seconds.

### I. BACKGROUND OF THE INVENTION

1. Field of the Invention

This invention pertains to treatments of disorders associated with neural activity. These may include, without limitation, gastrointestinal disorders (including obesity or bulimia) and pancreo-biliary disorders. More particularly, this invention pertains to treatment of such disorders through management of neural impulses.

2. Description of the Prior Art

The prior art describes treatments for a wide variety of disorders where the treatment includes blocking neural impulses on the vagus nerve. The blocking can be used as a therapy by itself or used in combination with traditional electrical nerve stimulation. The disorders to be treated include, without limitation, functional gastrointestinal disorders (FGIDs) (such as functional dyspepsia (dysmotility-like) and irritable bowel syndrome (IBS)), gastroparesis, gastroesophageal reflux disease (GERD), inflammation, discomfort and other disorders. Specific disorders to be treated include obesity and pancreatitis, each of which can be treated by down-regulating the vagus nerve. Such treatments are described in commonly assigned U.S. Pat. No. 7,167,750 to Knudson et al. issued Jan. 23, 2007 and in the following commonly assigned U.S. patent applications: US 2005/0131485 A1 published Jun. 16, 2005, US 2005/0038484 A1 published Feb. 17, 2005, US 2004/0172088 A1 published Sep. 2, 2004, US 2004/0172085 A1 published Sep. 2, 2004, US 2004/0176812 A1 published Sep. 9, 2004 and US 2004/0172086 A1 published Sep. 2, 2004.

The prior art literature includes disclosure of measuring pancreatic exocrine secretions (PES) as an indirect measurement of vagal activity. Increased PES production is an indicator of enhanced vagal activity. Decreased PES production is an indicator of inhibited vagal activity. An example of such a study for stimulating or inhibiting the vagus and measuring PES is Holst et al., “Nervous Control of Pancreatic Exocrine Secretion in Pigs”, Acta Physiol. Scand., Vol. 105, pp. 33-51 (1979). The Holst et al. article also suggests that stimulation of the splanchnic nerve can have the similar effect of a high frequency block applied to the vagus nerve. Namely, Holst et al. report that stimulating the splanchnic nerve decreases PES production in a manner similar to vagal down-regulation. International Patent Application Publication No. WO 2006/023498 A1 published Mar. 2, 2006 (filed in the name of applicant Leptos Biomedical, Inc., La Jolla, Calif.) purports to describe an obesity treatment involving stimulating a splanchnic nerve.

When applying an electrical signal to a nerve, the signal is commonly a series of pulses applied over a period of time. For example, to treat obesity, a down-regulating bi-polar signal is applied to both the anterior and posterior vagus nerves via electrodes placed on the nerves and connected to a pulse generator. As disclosed in U.S. patent application Publication No. US 2005/0038484 A1 published Feb. 17, 2005, the signal may be any signal in excess of a 200 Hz blocking signal reported by Solomonow, et al., “Control of Muscle Contractile Force through Indirect High-Frequency Stimulation”, Am. J. of Physical Medicine, Vol. 62, No. 2, pp. 71-82 (1983). A 5,000 Hz signal is currently most preferred. The current of the signal is selected to block the nerve without injury to the nerve. Such amplitudes may range from about 1 mA to 6 mA by way of non-limiting representative example.

Such signals are applied with a duty cycle. For example, U.S. Pat. No. 7,167,750 teaches applying a signal for five minutes (referred to herein as an “ON time”) followed by ten minutes of no signal (referred to herein as an “OFF time”). This pattern is repeated throughout the day (for example, while the patient is awake) and repeated for an indefinite number of days (e.g., daily for 6 months, 12 months or more).

It is an object of this invention to describe an optimized duty cycle of optimizing ON time and OFF time to maximize a therapeutic effect of a vagal down-regulation therapy.

### II. SUMMARY OF THE INVENTION

According to a method of treatment described in a preferred embodiment, a method is disclosed for treating a disorder (e.g., obesity) susceptible to treatment by applying an electrical signal to an autonomic nerve (e.g., a vagus or splanchnic nerve). The method includes applying a signal to a nerve of a patient to be treated. The signal has a duty cycle including an ON time during which the signal is applied to the nerve followed by an OFF time during which the signal is not applied to the nerve. The ON time is selected to have a duration preferably greater than 30 seconds and up to 180 seconds.

### III. BRIEF DESCRIPTION OF THE DRAWINGS

FIG. 1 is a schematic representation of an implantable system configuration for a gastro-intestinal treatment involving applying an electrical signal to a vagus nerve;

FIG. 2 is a scatter graph of patients treated with a vagal down-regulation procedure and showing excess weight loss (EWL) over time for each patient and showing a mean EWL;

FIG. 3 is a side elevation schematic view of an external coil in a desired alignment over an implanted coil;

FIG. 4 is the view of FIG. 3 illustrating misalignment of the external and internal coils resulting from changes in patient posture;

FIG. 5 is a graph illustrating percent excess weight loss over time experienced by patients grouped into visit interval-defined quartiles based on frequency of occurrence of ON times with durations less than 30 seconds;

FIG. 6 is a graph similar to that of FIG. 5 for patients grouped into visit interval-defined quartiles based on frequency of occurrence of ON times with durations between 30 and 180 seconds;

FIG. 7 is a graph similar to that of FIG. 5 for patients grouped into visit interval-defined quartiles based on frequency of occurrence of ON times with durations between 180 and 300 seconds;

FIG. 8 is a graph similar to that of FIG. 5 for patients grouped into visit interval-defined quartiles based on frequency of occurrence of ON times with durations between 30 and 120 seconds;

FIG. 9 is a graph similar to that of FIG. 5 for patients grouped into visit interval-defined quartiles based on frequency of occurrence of ON times with durations greater than or equal to 30 seconds;

FIG. 10 is a graph similar to that of FIG. 9 for patients grouped into subject-defined quartiles based on frequency of occurrence of ON times with durations greater than or equal to 30 seconds;

FIG. 11 is a graph illustrating efficacy as a function of therapeutic ON times;

FIG. 11A is a table illustrating the results of FIG. 11;

FIG. 12 is a graph illustrating patient response to the number of ON times experienced between follow-ups;

FIG. 13 illustrates an experimental set-up for studying effects of electrical signals on a nerve;

FIG. 14 is a graphs illustrating action potentials on a nerve;

FIG. 15 is a graph illustrating recovery of a nerve following a high frequency block; and

FIG. 16 is a graph illustrating a typical duty cycle.

### IV. DESCRIPTION OF A PREFERRED EMBODIMENT

The following commonly assigned patent and U.S. patent applications are incorporated herein by reference: U.S. Pat. No. 7,167,750 to Knudson et al. issued Jan. 23, 2007; US 2005/0131485 A1 published Jun. 16, 2005, US 2005/0038484 A1 published Feb. 17, 2005, US 2004/0172088 A1 published Sep. 2, 2004, US 2004/0172085 A1 published Sep. 2, 2004, US 2004/0176812 A1 published Sep. 9, 2004 and US 2004/0172086 A1 published Sep. 2, 2004. Also incorporated herein by reference is International patent application Publication No. WO 2006/023498 A1 published Mar. 2, 2006.

This application describes an optimized duty cycle for treating a wide variety of disorders. By way of non-limiting example, the invention is described in a preferred embodiment of a duty cycle for a down-regulating signal applied to the vagus nerve to treat obesity. The invention results from empirical analyses of data collected in an obesity study sponsored by the assignee of the present application.

A. Therapy Delivery Equipment

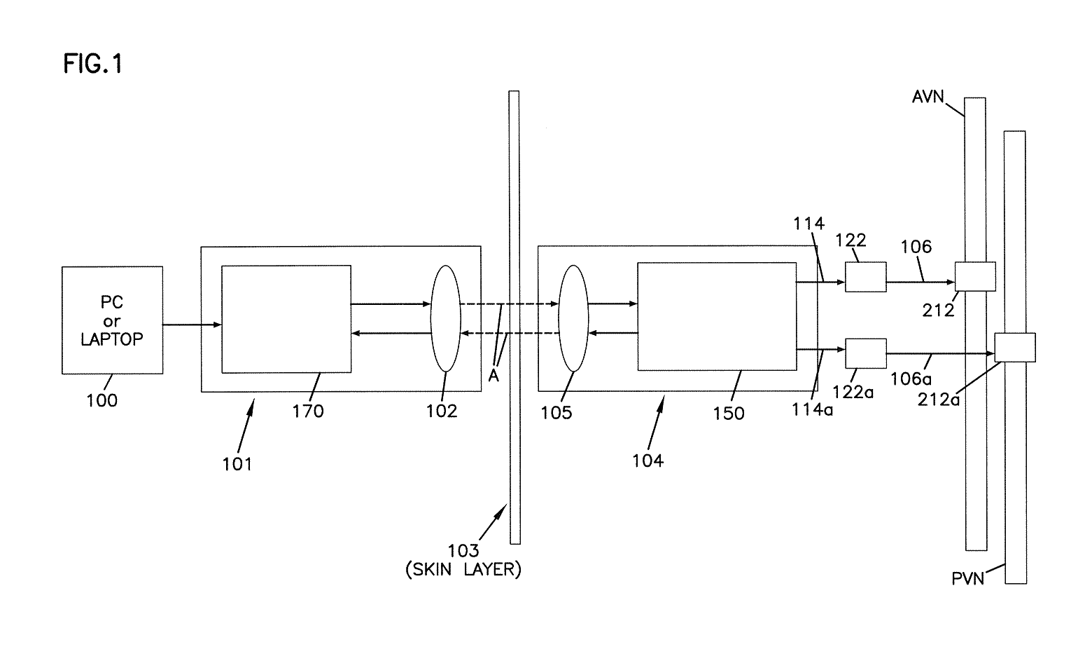

A system (schematically shown in FIG. 1) for treating obesity or other gastro-intestinal disorders includes a neuroregulator 104, an external mobile charger 101, and two identical electrical lead assemblies 106, 106a.

The neuroregulator 104 is adapted for implantation within a patient to be treated for obesity. The neuroregulator 104 is implanted just beneath a skin layer 103.

The lead assemblies 106, 106a are electrically connected to the circuitry of the neuroregulator 104 by conductors 114, 114a. Industry standard connectors 122, 122a are provided for connecting the lead assemblies 106, 106a to the conductors 114, 114a. As a result, leads 116, 116a and the neuroregulator 104 may be separately implanted. Also, following implantation, lead 116, 116a may be left in place while the originally placed neuroregulator 104 is replaced by a different neuroregulator.

The leads 106, 106a have distal electrodes 212, 212a which are individually placed on the anterior and posterior vagal nerves AVN, PVN, respectively, of a patient just below the patient's diaphragm. It will be appreciated that the description of two electrodes directly placed on a nerve is a description of a preferred embodiment. Fewer or more electrodes can be placed on or near fewer or more nerves.

The external mobile charger 101 includes circuitry for communicating with the implanted neuroregulator 104. The communication is a two-way radiofrequency (RF) signal path across the skin 103 as indicated by arrows A.

Referring to FIG. 1, a computer (such as a personal computer) 100 can be connected to the external mobile charger 101. With such a connection, a physician can use the computer 100 to program therapies into the neuroregulator 104 as will be described.

The circuitry 170 of the external mobile charger 101 can be connected to an external coil 102. The coil 102 communicates with a similar coil 105 implanted within the patient and connected to the circuitry 150 of the neuroregulator 104. Communication between the external mobile charger 101 and the neuroregulator 104 includes transmission of pacing parameters and other signals as will be described.

Having been programmed by signals from the external mobile charger 101, the neuroregulator 104 generates blocking signals to the bipolar leads 106, 106a. As will be described, the external mobile charger 101 may have additional functions in that it may provide for periodic recharging of batteries within the neuroregulator 104, and also allow record keeping and monitoring.

While an implantable (rechargeable) power source for the neuroregulator 104 is preferred, an alternative design could utilize an external source of power, the power being transmitted to an implanted module via the RF link (i.e., between coils 102, 105). In this alternative configuration, while powered externally, the source of the specific blocking signals could originate either in the external power source unit, or in the implanted module.

B. VBLOC-I Obesity Study

In early 2006, Assignee began a human pilot study (“VBLOC-I) to evaluate an obesity treatment according to the present invention. The inclusion criteria of the VBLOC-I study requires the patient have a body mass index (BMI) in a range between 35 and 50 (+/−10%). A BMI>30 is regarded as obese. A BMI>35 is generally regarded as morbidly obese.

After receiving the implant 104, the device is inactive for a two-week post-surgery healing period. Thereafter, the therapy is initiated. Patients are followed at regular periods throughout the study. The study is designed to measure efficacy at multiple time points post-implant. Efficacy is measured as the amount of excess weight loss (EWL) experienced by the patient. Excess weight is the difference between the patient's actual weight and ideal weight. The patient's excess weight is determined prior to surgery (“baseline”) as well as at multiple time points post-implantation. The EWL is the weight loss expressed as a percent of the baseline excess weight.

Patients enrolled in the VBLOC-I study receive an implantable component 104. All patients in the VBLOC-I study received an RF-powered version of the neuroregulator. The electrodes 212, 212a are placed on the anterior vagus nerve AVN and posterior vagus nerve PVN just below the patient's diaphragm.

The external antenna (coil 102) is placed on the patient's skin overlying the implanted receiving coil 105. The external control unit 101 can be programmed for various signal parameters including options for frequency selection, pulse amplitude and duty cycle. The frequency options include 2500 Hz and 5000 Hz (both well above a threshold blocking frequency of 200 Hz). The vast majority of treatments were at 5,000 Hz, alternating current signal, with a pulse width of 100 microseconds. The amplitude options are 1-6 mA. Duty cycle could also be controlled. A representative duty cycle is 5 minutes of blocking frequency followed by 5 minutes of no signal. The duty cycle is repeated throughout use of the device.

Normally a patient would only use the device while awake. The hours of therapy delivery can be programmed into the device by the clinician (e.g., automatically turns on at 7:00 AM and automatically turns off at 9:00 PM). In the RF-powered version of the neuroregulator, use of the device is subject to patient control. For example, a patient may elect to not wear the external antenna. The device keeps track of usage by noting times when the receiving antenna is coupled to the external antenna through radio-frequency (RF) coupling through the patient's skin.

C. Weight Loss Data

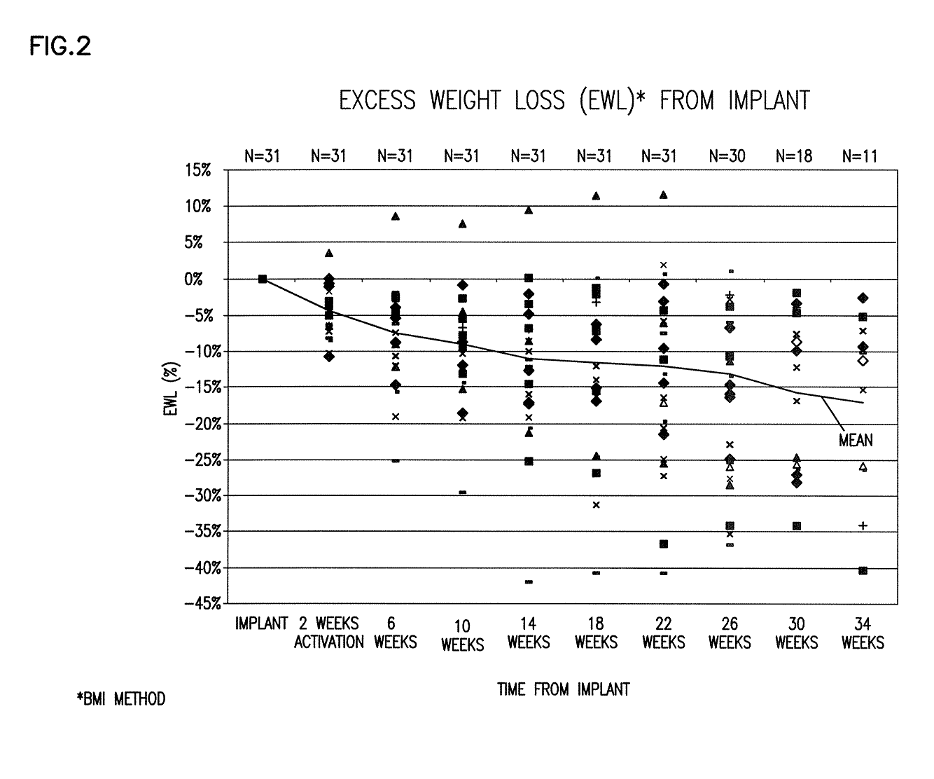

As would be expected in a human weight loss study, patients vary significantly in their response to treatment. However, overall weight loss has been very promising. Of thirty-one patients entered in the study, patients experienced an average weight loss of 16% after 34 weeks. FIG. 2 is an example of a scatter graph of all patients not otherwise excluded. “Excluded” means patient data was excluded for reasons of not using the device for significant periods or equipment failure. (E.g. Two patients are excluded from the data. Their exclusion is due to their extended periods of non-use of the device and questionable impedance data indicating therapy was not being delivered to the patient).

In FIG. 2, the vertical axis is the excess weight loss relative (as a percent of baseline weight). The horizontal axis is the number of weeks following treatment. “Maestro Implant” is the surgery date. “2 Weeks Maestro Activation” is the date of device activation following a 2-week post-surgery healing period. The remaining dates on the horizontal axis are post-surgery follow-up dates measured from date of surgery “Maestro Implant”.

The data are very encouraging. In any human treatment study, one expects patient-to-patient outcome variability. That is reflected in the data of FIG. 2. Applicants have analyzed the data to determine if the variability can be reduced or if the data otherwise permit useful conclusions top enhance therapy outcomes.

During the VBLOC-I study, patients were intended to receive a therapy dose of 5 minutes of electrical signal followed by 5 minutes of no signal. This duty cycle was to be repeated throughout the day.

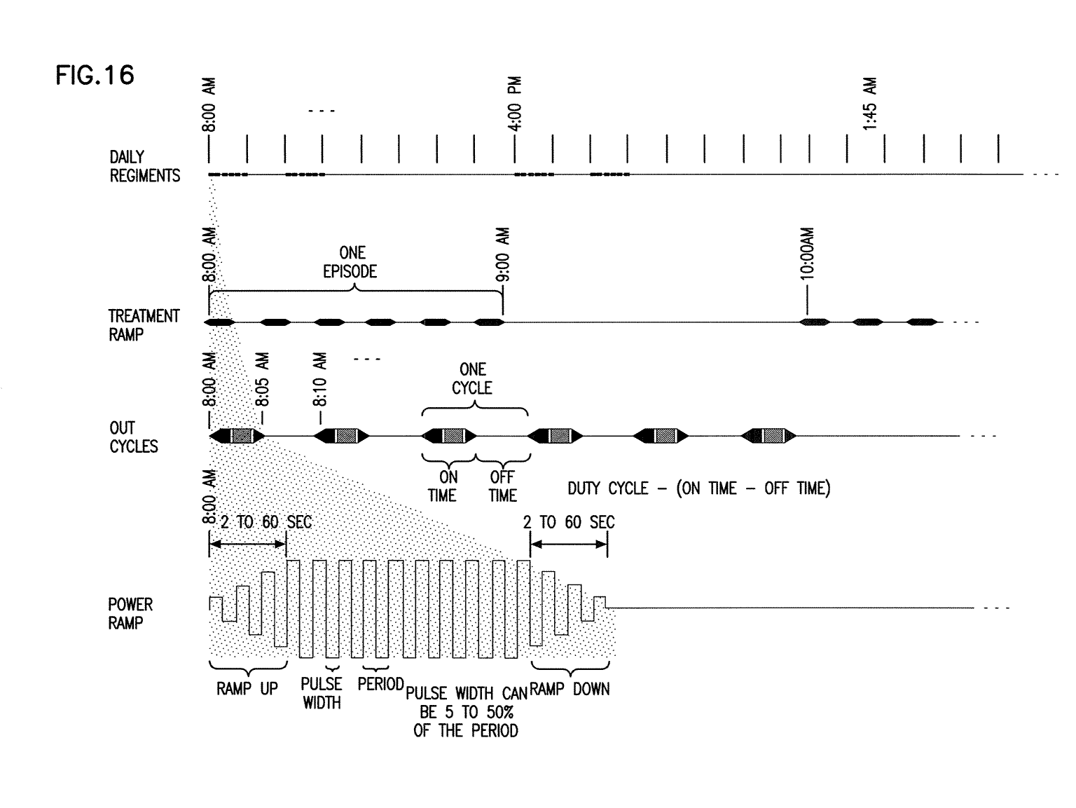

FIG. 16 shows a typical duty cycle. Each ON time includes a ramp-up where the 5,000 Hz signal is ramped up from zero amperes to a target of 6 mA. Each ON time further includes a ramp-down from full current to zero current at the end of the ON time. For about 50% of the patients, the ramp durations were 20 seconds and for the remainder the ramp durations were 5 seconds.

The use of ramp-ups and ramp-downs are conservative measures to avoid possibility of patient sensation to abrupt application or termination of a full-current 5,000 Hz signal. An example of a ramp-up for a high frequency signal is shown in U.S. Pat. No. 6,928,320 to King issued Aug. 9, 2005.

Not shown in the drawings, each ramp-up and ramp-down in the VBLOC-I study was broken into mini-duty cycles consisting of many imbedded OFF times of very short duration. While the mini-duty cycle was not completely uniform, it is approximated by 180 millisecond periods of mini-ON times of 5,000 Hz at a current which progressively increases from mini-ON time to mini-ON time until full current is achieved (or progressively decreases in the case of a ramp-down). Between each of such mini-ON times, there is a mini-OFF time which can vary but which is commonly about 20 milliseconds in duration during which no signal is applied. Therefore, in each 20-second ramp-up or ramp-down, there are approximately one hundred mini-duty cycles, having a duration of 200 milliseconds each and each comprising approximately 180 milliseconds of ON time and approximately 20 milliseconds of OFF time.

Analyzing data recovered during the post-surgery follow-ups, Applicants noted that, frequently, patients did not receive the full 5-minute dose. It was determined this was primarily due to loss of signal contact between the external controller 101 and implanted neuroregulator 104 due in large part to misalignment between coils 102, 105.

It is believed coil misalignment results from, at least in part, changes in body surface geometry throughout the day (e.g., changes due to sitting, standing or lying down). These changes can alter the distance between coils 102, 105, the lateral alignment of the coils 102, 105 and the parallel alignment of the coils 102, 105.

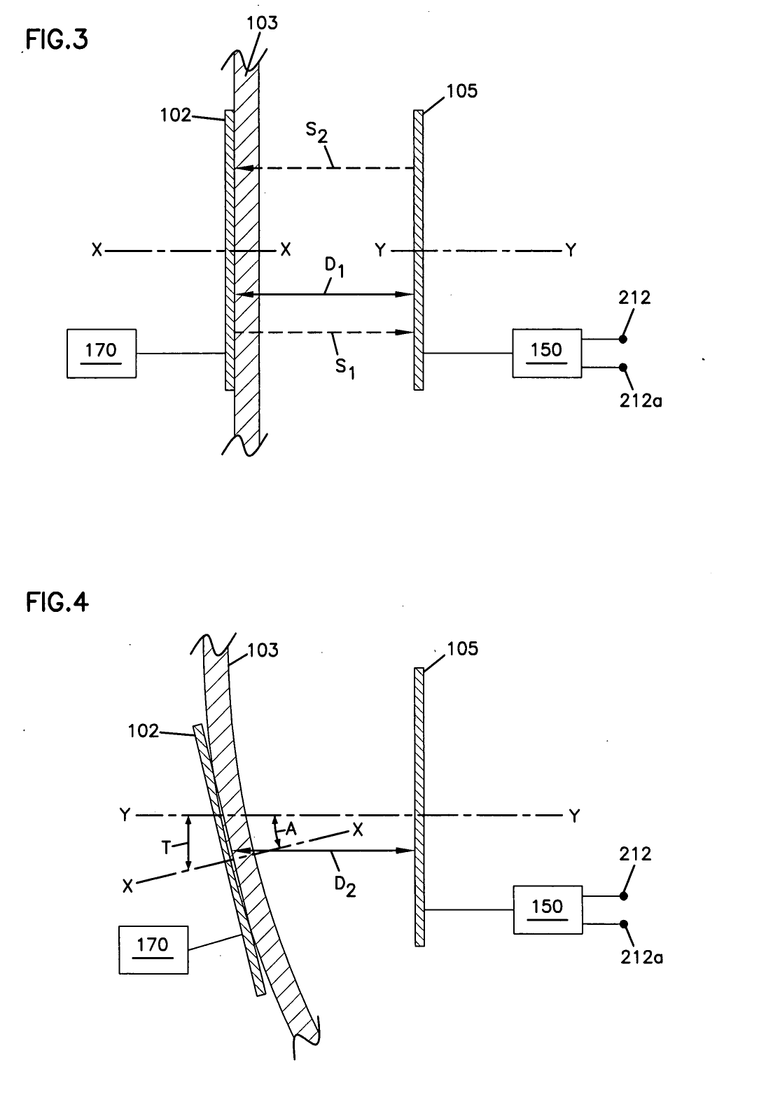

FIG. 3 illustrates a desired alignment. Coil 105 is implanted beneath the skin 103 at a preferred depth D₁ (e.g., about 2 cm to 3 cm beneath the skin 103), and with a plane of the coil 105 parallel to the surface of the skin 103.

Each coil 102, 105 is a circular coil surrounding a central axis X-X and Y-Y. As shown in FIG. 3, in an ideal alignment, the axes X-X, Y-Y are collinear so that there is no lateral offset of the axes X-X, Y-Y and the coils 102, 105 are parallel. Such an alignment may be attained when the external coil 102 is applied when the patient is lying flat on his back.

FIG. 4 illustrates misalignment between the coils 102, 105 resulting from posture changes. When the patient stands, excess fat may cause the skin 103 to roll. This increases the spacing between the coils 102, 105 to increase to a distance D₂. Also, the axes X-X and Y-Y may be laterally offset (spacing T) and at an angular offset A. These changes may be constantly occurring throughout the day.

As a result of coil misalignment, there may be a significant variance in the power received by the implanted coil 105. In the case of an implant receiving both power and command signals, in extreme cases, the power of a signal received by the implanted circuit 150 may be so weak or the communication link between the controller 101 and neuroregulator 104 may be so poor that therapy is lost.

Since such unintended signal interruption is undesirable, the assignee of the present application has developed improvements in design to reduce the likelihood of signal loss. Also, prior art coil alignments are described in U.S. patent applications Publication Nos. US 2005/0107841 to Meadows, published May 19, 2005, and US 2005/0192644 to Boveja, published Sep. 1, 2005. These applications teach alignment by measuring changes in reflected impedance and voltage.

D. Observed Variations In Duty Cycle

a. Length of ON Times

During patient follow-up visits in the VBLOC-I study, the external controller 101 can interrogate the implantable component 104 for a variety of information. From the collected data, Applicants can determine how often the patient is receiving the intended therapy. For example, Applicants can determine if a patient is receiving a full five minutes of an intended 5-minute therapy or only a portion (10 seconds, 1 minute, 4.5 minutes, etc).

Applicants had expected that patients receiving therapy for less than the maximum 5 minutes per duty cycle would be at a therapy disadvantage. However, after close analysis of the collected data, Applicants noted that within a narrow range of potential therapy per duty cycle, a range of actual therapy stood out as being surprisingly superior. Specifically, Applicants noted that therapy times of 30 seconds to 180 seconds per duty cycle were significantly superior to therapy times of less than 30 seconds per duty cycle or greater than 180 seconds per duty cycle. While Applicants do not fully understand the reason why such times are superior, the statistical data convince Applicants of the superiority.

b. Number of Therapeutic ON Times

During a 10 minute duty cycle (i.e., intended 5 minutes of therapy followed by a 5 minute OFF time), a patient can have multiple treatment initiations. For example, if, within any given 5-minute intended ON time, a patient experienced a 35-second ON time and 1.5 minute actual ON time (with the remainder of the 5-minute intended ON time being a period of no therapy due to signal interruption), the patient could have two actual treatment initiations even though only one was intended. The number of treatment initiations varies inversely with length of ON times experienced by a patient.

E. Statistical Analysis Of Duty Cycle Data and Weight Loss

Applicants performed a statistical analysis of collected data from the VLOC-I study. The goals of such analysis included understanding VBLOC-I efficacy data in order to optimize future use of the therapy.

The primary analysis method employed was a mixed model, repeated measures regression analysis. This methodology is standard for longitudinal or serially collected data. In the VBLOC-I study, data on delivered therapy (actual ON times) and excess weight loss (EWL) were available for at least some of the patients at weeks 1, 2, 3, 4, 6, 8, 10, 12, 16, 20 and 24 post-therapy initiation (with therapy initiation being 2-weeks post implantation).

Data from a particular subject patient across follow-up visits were correlated, and the mixed model regression analysis effectively accounted for this correlation and avoided the situation whereby the effect size of a particular parameter was overestimated. This analysis essentially computed an average effect for each subject and averaged that effect across subjects, weighted according to the amount of information each subject was contributing.

a. Quartile Analysis

To facilitate an analysis, patients were grouped into quartiles based on the number of ON times experienced by a patient. For example, for any given follow-up period (e.g., 6 weeks post-therapy initiation corresponding to 8 weeks post-implantation), twenty-four patients may report for such follow-up (the numbers given here are hypothetical for ease of explanation). Interrogation of the patients' implants reveal the patients have a wide number of different therapy initiations (correlating inversely with a wide variety of ON time durations). Patients are divided into quartiles based on the number of ON times experienced by the patient. In the example given, Quartile 4 would be the six patients (i.e., 25%) having the most number of ON times. Quartile 1 would the six patients (i.e., 25%) having the fewest number of ON times.

A quartile analysis can be made using, among other options, a visit interval-defined quartile analysis or a subject-defined quartile analysis. Applicants choose a visit interval-defined quartile analysis. However, information is supplied below showing comparability of such analysis with a subject-defined quartile analysis.

b. Visit Interval-Defined Quartile Analysis

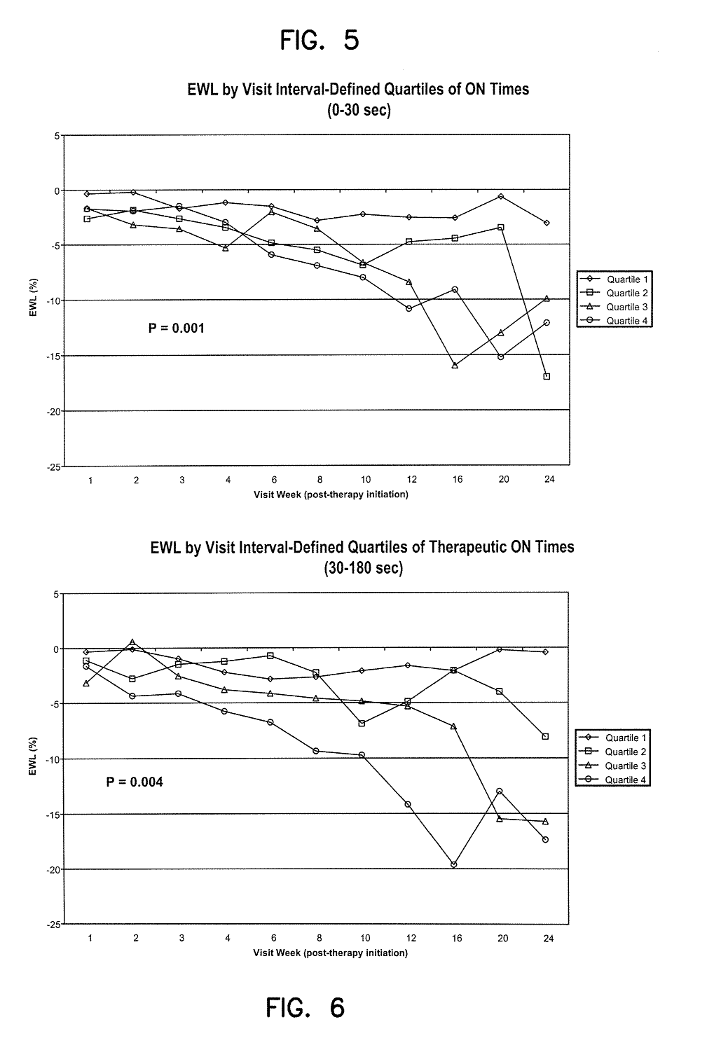

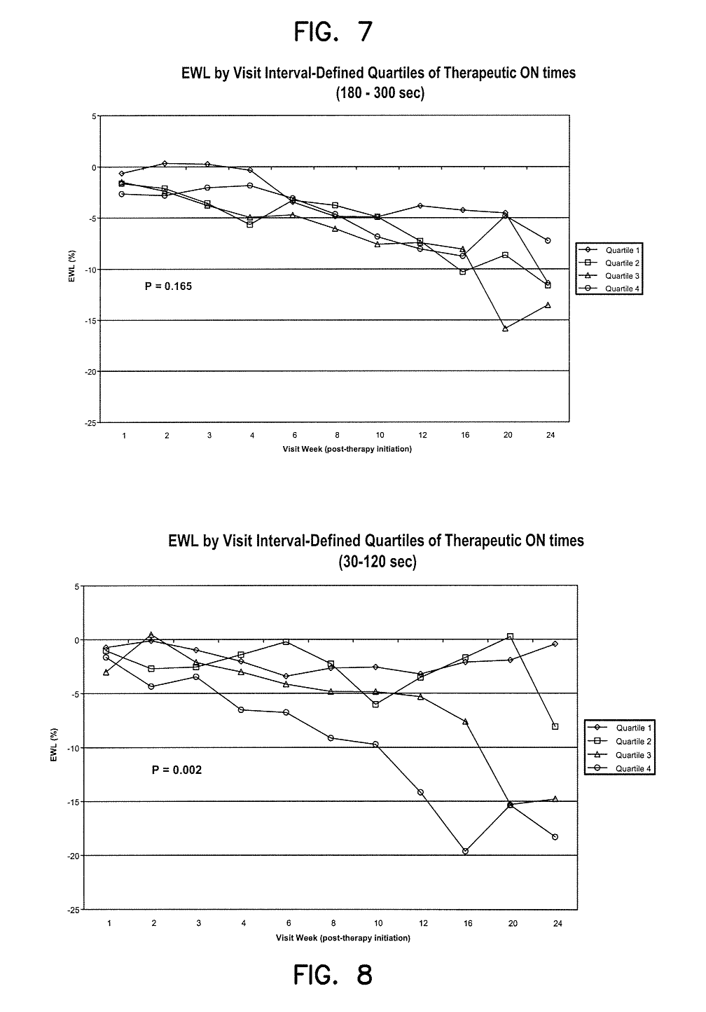

In FIGS. 5-7, therapeutic ON time quartiles are defined according to visit intervals. These figures illustrate the effect of the number of ON times of a specific duration. In these figures, discrete ON time durations (i.e. 0-30 seconds (FIG. 5), 30-180 seconds (FIG. 6), and 180-300 seconds (FIG. 7)) are analyzed in a repeated measures regression model to determine the duration of ON time with the greatest effect on EWL.

In FIG. 5, there is a relationship between quartiles and EWL as represented by the statistically significant “p-value” of 0.001. (A “p-value” of less than 0.05 is generally regarded as significant since it represents a 95% confidence level that the data variations are attributable to non-random events). However, the effect of this 0-30 second ON time is an order of magnitude less than that seen with therapeutic ON times of either 30-120 or 30-180 seconds (as discussed below) as shown by the parameter estimates of Table 11A.

In FIG. 6, there is a strong relationship between quartiles of therapeutic ON times from 30-180 seconds and EWL as evidenced by the p-value of 0.004. This therapeutic ON time duration of 30-180 seconds (which includes, as a subset, ON time durations of 30-120 seconds (FIG. 8)), represents the ON time with the greatest effect on EWL.

In FIG. 7, there is no statistically significant quartile effect of therapeutic ON times from 180-300 seconds as shown by the relatively high p-value of 0.165. The frequency of longer duration ON times is inconsequential in terms of incremental EWL. There is no additional benefit of longer ON times, relative to shorter ON times, with respect to EWL.

FIG. 8 analyzes a subset (30-120 seconds) of the data of FIG. 6 (30-180 seconds). As with the analysis of 30-180 second therapeutic ON times (FIG. 6), there is a strong relationship between quartiles of ON times from 30-120 seconds and EWL as evidenced by the p-value of 0.002. This therapeutic ON time duration of 30-120 seconds represents the optimal combination of effect on EWL (and battery longevity for a battery powered implant).

c. Study Subject-Defined v. Visit Interval-Defined Quartile Analyses

In a visit interval-defined quartile analysis, subjects are allowed to move from one quartile to another over the follow-up period. The repeated measures analysis described above adequately accounted for the visit-to-visit movement by an individual subject from one quartile to another by isolating the effect of ON times to the interval preceding each study visit and calculating a slope across visits.

By allowing movement between quartiles across visits, the analysis addressed the fact that ON times were not necessarily consistent across all visits for an individual study subject. For instance, if an intermittent or inconsistent link developed during an interval between visits but was then corrected at the next visit, that individual subject might have a greater number of therapeutic ON times (≧30 seconds) for the period of time with an inconsistent link compared with the period of time with consistent link. If ON times are associated with EWL, there would be a different effect on weight loss for the period of time with a greater frequency of therapeutic ON times compared with the period of time with consistent link.

Through the course of follow-up, that subject may have an average or low number of ON times and a different overall weight loss than was observed during the period of time with an inconsistent link. By allowing for movement across quartiles, we are able to account for such interval effects of ON times on EWL.

There is value, though, in also examining the cumulative frequency of therapeutic ON times through a certain follow-up visit (e.g. 20 weeks) and dividing subjects into quartiles according to the grand total number of ON times (corrected for total days on study). This analysis evaluates whether or not the cumulative (over 20 weeks) total number of therapeutic ON times has an effect on excess weight loss. The repeated measures approach in this instance adjusts for the within-patient correlation across follow-up visits, but does not take into account that a subject may have a variable frequency of ON times from one visit to another. That is, only the average frequency of ON times over the course of follow-up is considered. This type of analysis is “study subject-defined quartile analysis”.

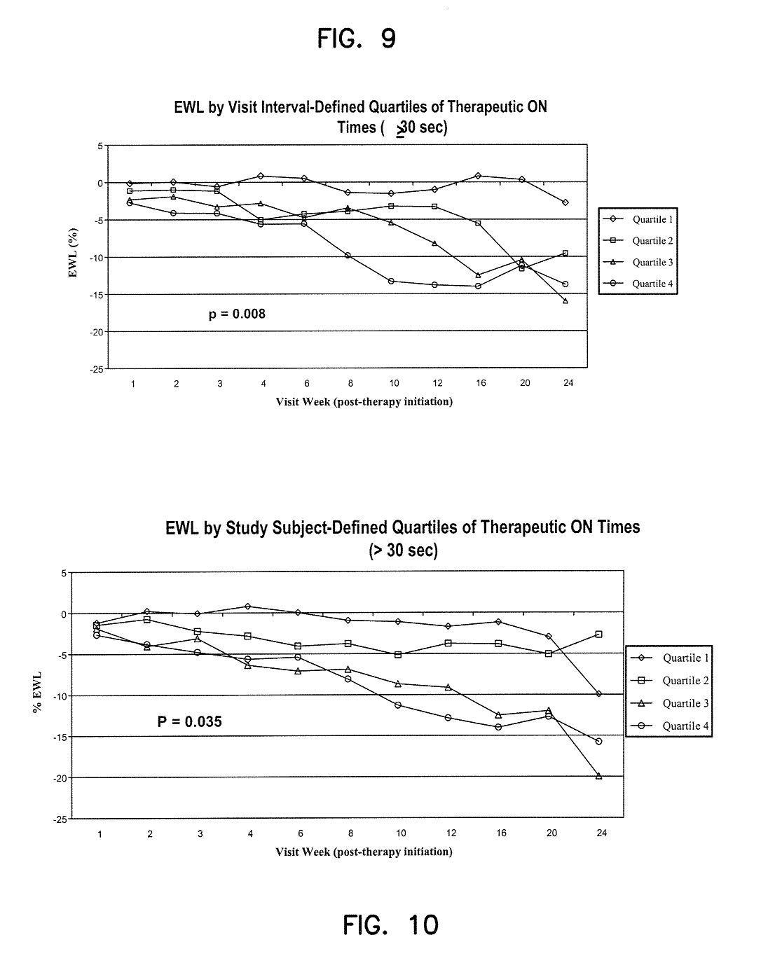

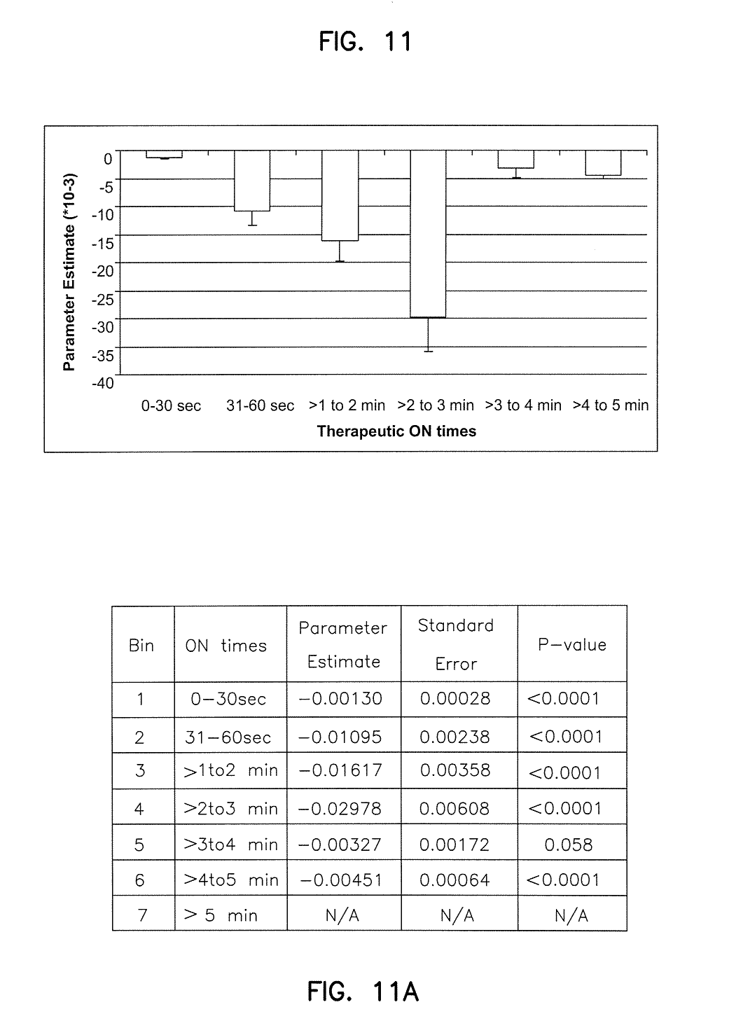

Study subject-defined and visit interval-defined quartile analyses are compared in FIGS. 9 and 10. In these analyses, “ON time” means an actual therapy time greater than or equal to 30 seconds. Quartiles are divided on the basis of frequency of ON times.

The p-value in these analyses is the significance of the effect across quartiles.

This p-value not only incorporates a measure of linearity, but also effect size. A non-significant p-value would be an indication of no linear effect of therapeutic ON times on % EWL.

A similar effect is seen in both analyses. There is a generally linear effect of the number of ON times (according to quartile) and the percent EWL. The significance level for both analyses is statistically significant, though the more granular analysis (visit interval-defined quartiles) is more significant. Because the patient groups for the study subject-defined quartiles is determined according to the cumulative number of ON times over a fixed period of time (20 weeks), sample size is smaller (29 vs. 31 subjects) as data was not available at 20 weeks for two subjects.

Defining quartiles in the described manners yield similar results in terms of the effect of therapeutic ON times on excess weight loss. Evaluating subject-defined quartiles has confirmed the findings from the study visit interval-defined quartile analysis.

From a comparison of FIGS. 9 and 10, Applicants conclude the mixed model, repeated measures regression models are appropriate for both quartile-defined analyses. A strong, linear relationship exists between frequencies of therapeutic ON times greater than or equal to 30 seconds and excess weight loss in the VBLOC-I study population. Each of the two quartile analyses yield consistent results and conclusions, and are mutually confirmatory

d. Additional Analysis

FIGS. 11 and 11A graphically illustrate an alternative analysis showing the observed superiority of 30 to 180 seconds therapy per duty cycle versus other options within a 0 to 5 minute range. FIGS. 11 and 11A represent the parameter estimates associated with distinct ON time bins. A “bin” is an assignment of data. For example, “Bin 1” is defined as data associated with ON times of less than 30 seconds. The bins are reflected in Table 11A.

FIGS. 11 and 11A represent the parameter estimates associated with distinct ON time bins. Bins are retrospective groupings to permit analyzing the correlation, if any, between length of ON times and excess weight loss.

For each bin, a parameter estimate is given. These parameter estimates are from a mixed model, repeated measures regression analysis that estimates the effect of the cumulative number of ON times of a given duration over time. Such models and analyses are well known in statistics.

The parameter estimate represents the slope of the regression line, and a one-unit increase in the cumulative number of ON times for a particular bin is associated with a percent of excess weight loss equal to the parameter estimate for that ON time. For example, a 100 unit increase in the number of ON times from two to three minutes in duration is associated with a −2.9% EWL.

G. Conclusions from Statistical Analysis

From the foregoing, Applicants conclude a greater number of initiations of therapeutic ON times during any given time period are associated with greater excess weight loss (EWL). This therapeutic effect is greatest with therapeutic ON times of either 30-180 seconds (p=0.004) or 30-120 seconds (p=0.002). Therapeutic ON time durations of 30-120 seconds represent the optimal combination of effect on EWL and battery longevity.

Applicants do not, at present, thoroughly understand why 30 to 180 seconds shows superior results. As a matter of conjecture, the central nervous system may accommodate to a loss of vagal neural activity after about 180 seconds, or accommodation may be due to membrane changes and local accommodation.

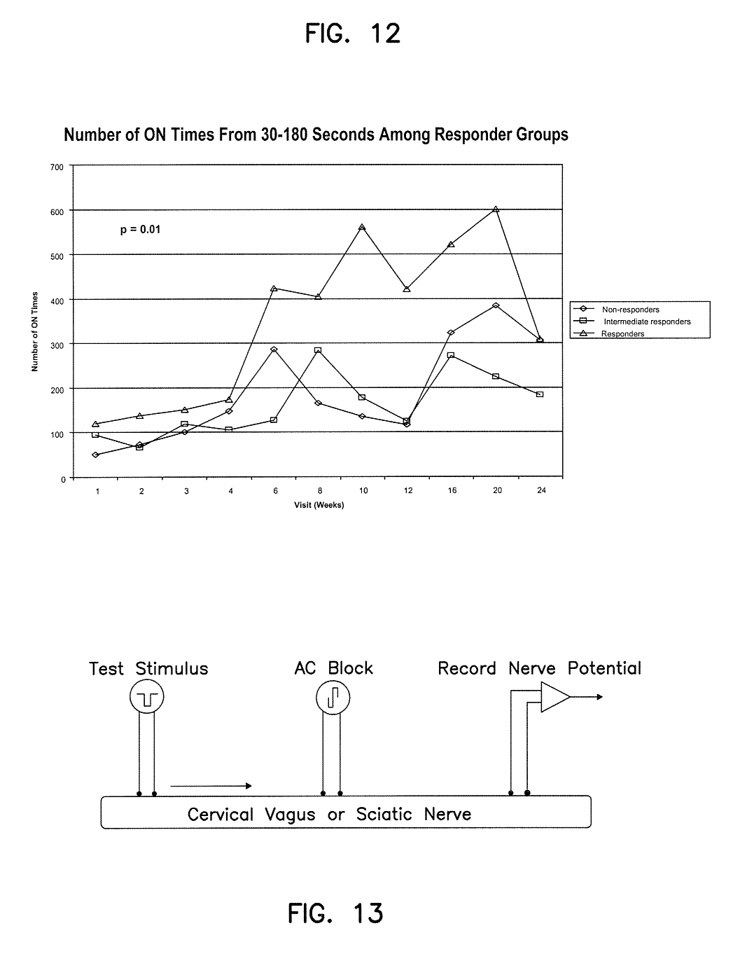

In addition to a preferred ON time of 30 seconds to 180 seconds, the duty cycle preferably has a short OFF time to maximize the number of initiations of such duty cycles per day. FIG. 12 graphically illustrates patient response to the therapy based on the number of ON times experienced by the patient. For FIG. 12, “ON time” means only those treatment durations between 30 to 180 seconds. If the patient experienced additional treatments of different durations (e.g., less than 30 seconds or greater than 180 seconds), those additional treatments are ignored in FIG. 12.

In FIG. 12, the horizontal axis is the number of week's post-activation of the implant. The vertical axis is the number of treatment ON times (again, defined for the purpose of FIG. 12 as between 30 and 180 seconds) experienced by the patient between follow-up visits.

It should be noted that not the same number of patients are in the data points for each horizontal axis location. Since patients are implanted over a period of time, while all patients had early follow-ups at the time of the analysis, not all such patients had later follow-ups. Therefore, there are more data for early weeks than for later weeks. This is also true for the other graphs described in this application.

In FIG. 12, patients are grouped into groupings labeled “non-responders”, “intermediate responders” and “responders”. For the purpose of FIG. 12, “non-responders” is defined as patients who experience an excess weight loss of less than or equal to zero (includes patients who gained weight). “Intermediate responders” is defined as patients who experience an excess weight loss greater than zero and less than or equal to 10%. “Responders” is defined as who patients experience an excess weight greater than 10%.

FIG. 12 further supports the surprising conclusion that 30 to 180 seconds is a preferred ON time of a duty cycle. Responders have many more such ON times than non-responders or intermediate responders. In addition, FIG. 12 may suggest the duty cycle should include an OFF time (period of time when a signal is not applied to the nerve) that is short in duration in order to maximize the number of such 30-to-180 second ON times per day.

The OFF time should be long enough to permit at least partial recovery of the nerve from the effect of the ON time. Applicants' data suggest that an OFF time period less than five minutes and, more preferably, less than two minutes permits partial recovery. By way of non-limiting examples, improved duty cycles may be (1) 2-minutes ON followed by 1-minute OFF followed by 2-minutes ON followed by 5 minutes OFF or (2) 1.75-minutes ON followed by 1-minute OFF followed by 2.5-minutes ON followed by 5 minutes OFF. These examples illustrate techniques to increase the number of ON times per day and also illustrate the duration of ON times need not be uniform. For example, the duration could be randomly distributed within the preferred range (30 to 180 seconds).

Specifically, Applicants have studied the effect of blocking frequencies and recovery times on rat nerves. FIG. 13 illustrates an experimental set-up. A rat's cervical vagus nerve or sciatic nerve is isolated to be used as a test nerve for study. Three bipolar hook electrodes are placed in series on the isolated nerve. A first electrode (labeled “Test Stimulus” in FIG. 13) is a generating electrode for generating a stimulation signal (i.e., inducing a propagating neural impulse or compound action potential (“CAP”)). A second electrode (labeled “AC Block” in FIG. 13) applies a neural blocking signal (e.g., a series of alternating current pulses with a frequency in excess of a threshold blocking frequency of 200 Hz). A third electrode (labeled “Record Nerve Potential” in FIG. 13) connects the nerve to recording equipment to record neural impulses.

With the experiment of FIG. 13, a stimulating signal (a series of electrical pulses applied at a frequency below a 200 Hz blocking threshold) is applied to the first electrode. A blocking signal (greater than 200 Hz) is applied to the second electrode for a period of time. After such period, the nerve impulses can be recorded by the third electrode. The frequency and duration of the blocking signal at the second electrode are varied to observe the effect of such variables on the recorded response at the third electrode.

The amplitude of evoked fast and slow CAP waves was measured (at the third electrode) before and after applying blocking pulses of selected frequency and duration. Post-block measurements were taken at time points (e.g., 0-5, 10 and 15 minutes) after discontinuing the blocking signal.

The graph of FIG. 14 shows normal (i.e., not subject to a blocking frequency) nerve response to a stimulation signal (i.e., less than 200 Hz). The nerve includes three types of nerve fibers designated Aαβ, Aδ and C fibers. The Aαβ and Aδ fibers are myelinated while the C-fibers are not myelinated. Being myelinated, the Aαβ and Aδ fibers have faster neural impulse propagation.

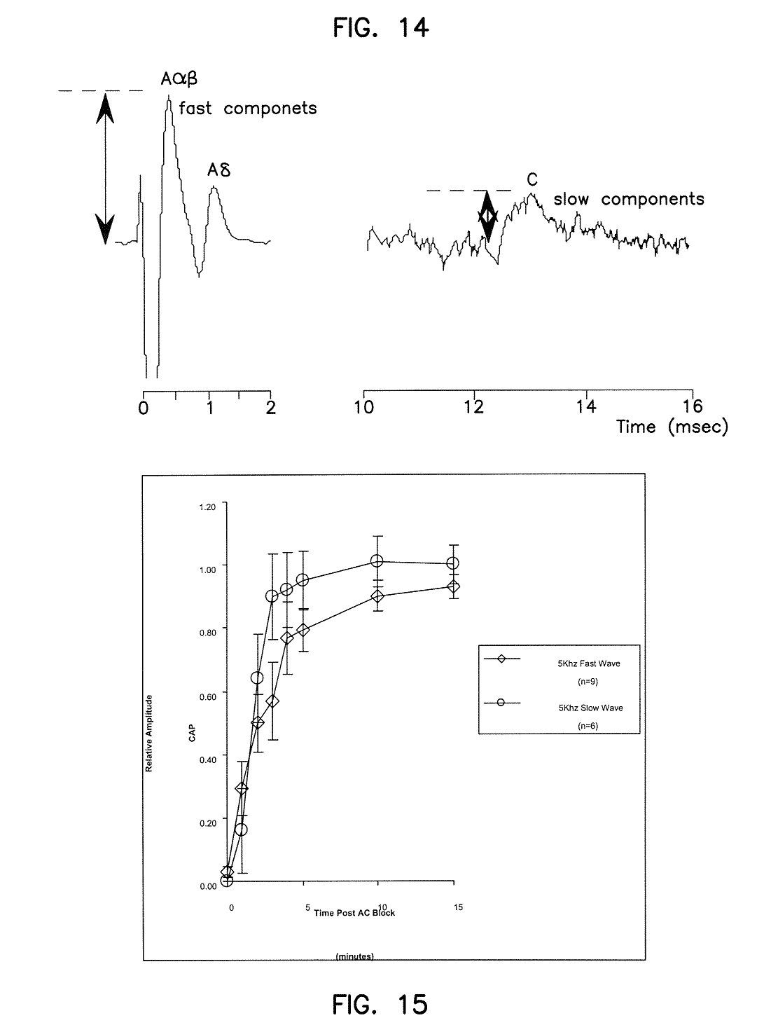

The graph of FIG. 15 shows fast and slow wave components after application of a blocking signal of 5,000 Hz for 5 minutes. FIG. 15 shows that fast and slow components were blocked at 5,000 Hz and 1 mA-4 mA. The graph also shows CAP recovery of 50% within two minutes post-block and by 90% within 10 minutes post-block.

From the above, an OFF time duration of less duration permits at least partial recovery of the nerve. Therefore, a short OFF time duration is preferred to maximize the number of ON times experienced by a patient per day while still permitting partial recovery of the nerve.

H. Ramp-Ups and Ramp-Downs

As a consequence of the shortened ON times from a target of 5-minutes, not many patients in the VBLOC-I study received any ramp-down. Only those experiencing an uninterrupted 5-minute ON time received a ramp-down. Further, patient treatments with actual ON-times less than 20-seconds in duration, never received treatment other than the mini-duty cycle ramp-up described above. Treatment durations greater than 20 seconds received a full ramp-up described above.

From the data, Applicants conclude that ramp-ups and ramp-downs are not beneficial from an efficacy perspective. For patients groups receiving the longest actual ON times (e.g., “>4 to 5 min” in FIG. 11), these include the only patients to receive a ramp-down. These patients experienced some of the worst efficacy correlation. Similarly, for patients for whom the ramp-up was the highest percent of the total ON time (group “0-30 sec” in FIG. 11), efficacy correlation was also poor.

With the foregoing detailed description of the present invention, it has been shown how the objects of the invention have been attained in a preferred manner. Modifications and equivalents of disclosed concepts such as those which might readily occur to one skilled in the art are intended to be included in the scope of the claims which are appended hereto. For example, while the foregoing is described with reference to applying blocking signals to vagus nerves to treat obesity, the invention is applicable to any disorder amenable to treatment by down-regulating the vagus nerve. Further, the invention is applicable to any blocking frequency applied to an autonomic nerve. Further, the invention is applicable to duty cycles for stimulating splanchnic nerves.

What is claimed is:

1. A method of treating a disorder susceptible to treatment by applying an electrical signal to an autonomic nerve, the method comprising:
applying a signal to a nerve of a patient to be treated for a disorder with the signal having a duty cycle including an ON time during which the signal is applied by to the nerve followed by an OFF time during the signal is not applied to the nerve; and
wherein the ON time is selected to have a duration no greater than 180 seconds.

2. A method according to claim 1 wherein the ON time is selected to have a duration no less than 30 seconds.

3. A method according to claim 1 wherein the OFF time is selected to have a duration for the nerve to at least partially recover to a baseline state following discontinuance of the OFF time.

4. A method according to claim 1 wherein the disorder is obesity.

5. A method according to claim 4 wherein the nerve is a vagus nerve and the signal is selected to down-regulate the nerve during the ON time.

6. A method according to claim 4 wherein the nerve is a splanchnic nerve and the signal is selected to up-regulate the nerve during the ON time.
